(19) United States
(12) Patent Application Publication
(10) Pub. No.: US 2026/0233868 A1
(43) Pub. Date: Aug. 13, 2026

(54) Method and Device for Producing a Bag with a Butt-Welded Connection

(71) Applicant: Fresenius Medical Care Deutschland GmbH, Bad Homburg (DE)

(72) Inventors: Pascal Joachimsky, Bad Homburg (DE); Andreas Klemm, Bad Homburg (DE); Franz Kugelmann, Bad Homburg (DE); Christian Schley, Bad Homburg (DE)

(21) Appl. No.: 19/153,266

(22) PCT Filed: Jan. 31, 2024
(86) PCT No.: PCT/EP2024/052400

§ 371 Date: Aug. 1, 2025

(30) Foreign Application Priority Data
Feb. 2, 2023 (DE) 102023102595.3

(51) Int. Cl. B65B 3/02; A61J 1/10; A61J 1/14; A61M 1/16

(52) U.S. Cl. CPC B65B 3/02; A61J 1/10; A61J 1/1462; A61M 1/1668; A61M 2207/00

(57) ABSTRACT

A method for producing a bag for at least one of receiving a medical productor a dry medical concentrate, includes: providing a flat film with two outer edges; fixing at least one connection to the flat film using butt welding; producing a film tube out of the flat film using a forming shoulder and by sealing the outer edges of the flat film together; producing a sealing seam that extends perpendicular to at least one of the longitudinal direction or the extending direction of the film tube, whereby a receiving pocket is produced in the film tube; filling the receiving pocket along at least one of the longitudinal direction or the extending direction of the film tube with liquid and/or solid contents; and closing the receiving pocket by producing an additional sealing seam that extends perpendicular to the longitudinal direction and/or the extending direction of the film tube, whereby the receiving pocket is closed and a completely closed chamber is formed.

### CROSS-REFERENCE TO RELATED APPLICATIONS

The present application is the national stage entry of International Patent Application No. PCT/EP2024/052400, filed on Jan. 31, 2024, and claims priority to Application No. DE102023102595.3, filed in the Federal Republic of Germany on Feb. 2, 2023, the entire contents of which are hereby incorporated by reference.

### TECHNICAL FIELD

The present disclosure relates to a method and a device for producing a bag with at least one butt-welded connection.

In particular, the present disclosure relates to bags from the medical field, which are used, for example, for storing medical solutions, e.g., dialysate or a partial component of dialysate, such as a concentrate. Other examples of medical solutions are aqueous solutions of sodium chloride, sodium citrate or calcium chloride as well as preparations for parenteral nutrition.

### BACKGROUND

Traditionally, bags in the medical sector are made from two layers of film, which are sealed to form a bag. Connectors, connections, or ports of the bags are usually interposed between the two layers of film. The first step is to separate the two layers of film from each other, then the connections are inserted and the films are welded together.

In particular, leaks or weak points often occur in practice at the so-called triple points, which represent a transition between the two films and the connection. In addition, welding a three-dimensional contour, such as the one formed by the intermediate end, requires complex tools. Furthermore, thin spots often occur in the film in the area of such sealed connections, which can lead to weak points in the bag.

### SUMMARY

An advantage of the present disclosure is to mitigate or even completely eliminate the problems of the prior art. In an example, an advantage of the present disclosure is to provide a method and a device in which bags from the medical sector can be equipped with connections in a simple, durable, and cost-effective manner.

Accordingly, a method for producing a bag for receiving a medical product, e.g., a medical solution, e.g., dialysate, and/or a dry medical concentrate is provided, the method including:
a) providing a flat film with two outer edges;
b) fixing at least one connection to the flat film by butt welding;
c) producing a film tube out of the flat film by a forming shoulder and by sealing the outer edges of the flat film together;
d) producing a sealing seam that extends perpendicular or substantially perpendicular to a longitudinal direction and/or extending direction of the film tube, whereby a receiving pocket is produced in the film tube;
e) filling the receiving pocket along the longitudinal direction and/or the extending direction of the film tube, e.g., in the vertical direction from above, with liquid and/or solid contents; and
f) closing the receiving pocket by producing an additional sealing seam that extends perpendicular or substantially perpendicular to the longitudinal direction and/or the extending direction of the film tube, whereby the receiving pocket is closed, and a completely closed chamber is formed.

In the broadest sense, steps a) and c) to f) are optional and a method for producing a bag can include at least one connection of the bag that is arranged or fixed to the bag by butt welding according to step b).

Attaching the connection to the film by butt welding can offer the advantage that the connection sits on an outermost layer of the folded-over film and is not integrated into the film or its layer structure or welded between two layers of film that form the bag.

In some embodiments, the term butt welding refers to a method in which two end faces are butt welded together in contact with each other. Butt welding is also known as mirror welding.

For example, the butt-welded connection can have a tube section. Firstly, the tube section is widened, e.g., flange-like or tulip-shaped, at one end of the tube section in order to create a larger end face in the manner of a flange. The end face of the tube section is then butt-welded to a surface, such as a film, and welded to it under pressure and heat.

In this procedure, the end face of the tube section then sits on the surface of the film and is not integrated into it or interposed between two films.

Butt welding can be used to create strong and resilient joints.

A plurality of connections can be arranged on a bag, of which at least one, e.g., all, is/are arranged by means of butt welding on the film, e.g., on a side formed by a folded-over film or on the edge of the bag.

According to one embodiment, step b) of a method comprises at least one of the following steps:
providing a cylindrical tube section;
preforming a connection section of the tube section by locally expanding the tube, e.g., at one end area of the tube section, whereby a flange-like connection section is preferably formed by means of pressure and heat;
placing the connection section on the flat film so that the flat film is interposed between the connection section and a pressure plate; and
welding the connection section to the flat film by pressing on the connection section while exerting a counter-pressure by means of the, e.g., heated pressure plate, whereby the connection section is connected to the flat film and forms a connection.

The cylindrical tube section can have an outer diameter (length a in FIG. 1) of 5 mm to 10 mm, e.g., of 4.9 mm to 8 mm and an inner diameter (length b in FIG. 1, diameter of the lumen) of 3 mm to 8 mm, e.g., of 3.5 mm to 6 mm.

According to one embodiment, in step b), a connection section is preformed at a temperature of between 80° C. and 300° C., e.g., between 220° C. and 280° C., for a period of time of 0.1 s to 4 s, e.g., from 1.5 s to 3.5 s, and at a pressure of 0.1 N/mm² to 2 N/mm², e.g., from 0.3 N/mm² to 0.5 N/mm². This is illustrated in the table below:

### Parameters for Preforming:

| | |
|---|---|
| Welding temperature: | 80° C. ≤ T ≤ 300° C. |
| Welding time: | 0.1 s ≤ t ≤ 4 s |
| Welding pressure: | 0.1 N/mm² ≤ σ ≤ 2 N/mm² |

In some embodiments:

| | |
|---|---|
| Welding temperature: | 220° C. ≤ T ≤ 280° C. |
| Welding time: | 1.5 s ≤ t ≤ 3.5 s |
| Welding pressure: | 0.3 N/mm² ≤ σ ≤ 0.5 N/mm² |

Furthermore, e.g., in step b), a flange-like connection section of a tube section is formed during preforming, wherein a thickness of the flange-like connection section (length c in FIG. 2) in the longitudinal direction of the tube section is between 0.1 mm and 2 mm, e.g., between 0.3 mm and 0.5 mm, and e.g., is approximately 0.4 mm, and a diameter of an end face of the flange-like connection section (length d in FIG. 2) is between 5 mm and 25 mm, e.g., between 10 mm and 14 mm, and e.g., is 12 mm.

In addition, in step b) a connection section can be placed on the flat film, wherein a temperature of the connection section, e.g., the flange-like connection section, is at least partially between 200° C. and 280° C.

A transfer time from a first position, in which the connection section is initially spaced apart from the film, to a second position, in which the connection section is seated on the flat film, is between 0.1 s and 1 s. After the connection section has been preformed, it is therefore placed on the film quite quickly and then welded thereto.

In step b) welding of the connection section to the flat film can be carried out at a temperature of the pressure plate of 80° C. to 150° C., e.g., 90° C. to 110° C. for a time period of 0.1 s to 4 s, e.g., 1.5 s to 3.5 s and a pressure of 0.1 N/mm² to 2 N/mm², e.g., 0.3 N/mm² to 0.5 N/mm².

In other words, the parameters for butt welding according to step b) can be as follows:

### Placement Temperature of Connection Section (Residual Heat)

| | |
|---|---|
| Welding temperature: | 200° C. ≤ T ≤ 280° C. |
| Welding time: | 0.1 s ≤ t ≤ 4 s |

### Heated Pressure Plate Film

| | |
|---|---|
| Welding temperature: | 80° C. ≤ T ≤ 150° C. |
| Welding time: | 0.1 s ≤ t ≤ 4 s |
| Welding pressure: | 0.1 N/mm² ≤ σ ≤ 2 N/mm² |

In some embodiments:

| | |
|---|---|
| Welding temperature: | 90° C. ≤ T ≤ 110° C. |
| Welding time: | 1.5 s ≤ t ≤ 3.5 s |
| Welding pressure: | 0.3 N/mm² ≤ σ ≤ 0.5 N/mm² |

The following parameters can also be used when welding film to film, for example to form sealing seams or a peel seam:

| | |
|---|---|
| Welding temperature: | 150° C. ≤ T ≤ 300° C. |
| Welding time: | 0.7 s ≤ t ≤ 2 s |
| Welding pressure: | 0.1 N/mm² ≤ σ ≤ 2 N/mm² |

The following parameters are used in some embodiments:

| | |
|---|---|
| Welding temperature: | 190° C. ≤ T ≤ 250° C. |
| Welding time: | 0.9 s ≤ t ≤ 1.5 s |

Welding pressure: 3000 N where A=6300 mm²→0.48 N/mm²

### Tensile Tests

The stability of the butt-welded connection between the bag film and the tube was tested using tensile tests on a universal testing machine. For this purpose, 10 test samples were produced and measured. The tensile tests were repeated 10 times each.

The results of the tensile tests are summarised in Table 1.

### Legend

FT=failure of tube
FF=failure of film
FWP=failure of welding point
FCP=failure of clamping point

TABLE 1
Results of tensile tests
CAPD-CD-ZP-AV-V

| Test | Max. tensile | Elongation at | |
|---|---|---|---|
| sample | force [N] | break | Type of failure |
| F-1 | 118.68 | 336.18 | FCP |
| F-2 | 107.1 | 307.01 | FCP |
| F-3 | 105.5 | 285.66 | FF |
| F-4 | 110.62 | 299.21 | FF/FT |
| F-5 | 106.08 | 283.51 | FCP |
| F-6 | 87.78 | 188.77 | FF/FT |
| F-7 | 110.62 | 298.45 | FCP |
| F-8 | 107.7 | 291.3 | FCP |

For particularly efficient bag production, completely closed chambers can be produced continuously along a continuous film tube, in each case connected to each other via a sealing seam, which chambers are separated from each other by separation along a sealing seam extending perpendicular or substantially perpendicular to the longitudinal direction and/or extending direction of the film tube, whereby separate bags are produced, each having one or more chambers.

In other words, a plurality of bags can be produced on one roll or film tube.

According to one embodiment, a bag produced using a method can be a multi-chamber bag.

According to this embodiment, a peel seam extending in the longitudinal direction is also, e.g., produced in the film tube, which peel seam detachably connects two layers of the film tube to one another, whereby in step d) two adjacent receiving pockets are produced, which are separated by the peel seam and which in step e) can be each filled by two separate filling lances with a liquid, solid and/or pasty content, which is designed for producing dialysate.

Another aspect of the present disclosure relates to a device fixing at least one connection of a film by means of butt welding, having:
a retaining device for holding a tube section with a, e.g., flange-like connection section;
a receiving section, in which the film can be positioned in contact with the contact section; and
a movable and, e.g., heatable pressure plate, wherein
a control unit is also provided, which is programmed to control the device to carry out a method as described herein.

In some embodiments, the control unit is programmed to move and control the retaining device and/or the pressure plate relative to each other in such a way that a connection section held in the retaining device is pressed together with a film positioned in the receiving section for a certain period of time and at a certain temperature and pressure, so that the connection section is butt-welded to the film and forms a connection.

Another aspect of the present disclosure relates to a bag, which is produced according to a method described herein and/or with a device described herein, and has at least one side which is formed by a folded-over film to which at least one connection, e.g., a tube section, is non-detachably arranged by means of butt welding.

The bag can be a multi-chamber bag with at least two chambers, which are separated from each other by an openable peel seam.

In some embodiments, the bag does not have, in addition to the at least one connection arranged on the bag by means of butt welding, a, e.g., non-detachably closed filling port designed to fill the bag or at least one chamber thereof with a desired content at the factory, e.g., before the bag is delivered to the end user.

Such a filling port can be understood as an access to a receiving area of the bag, which is designed to fill the receiving area of the bag before the bag or a bag system comprising the bag is delivered to an end user or before the bag is used in peritoneal dialysis, for example. After filling, the filling port is usually closed permanently, for example with a sealing cap.

The present disclosure also relates to a bag system for peritoneal dialysis with a solution bag and a drainage bag, which are connected to a common three-way valve, wherein the solution bag and/or the drainage bag is a bag according to the invention.

In some embodiments, steps a) to f) of a method are carried out with a single machine.

The present disclosure is not limited to the explicitly disclosed combinations of features, but that the features disclosed herein may also be claimed in other combinations or in isolation.

Further advantages, effects, and features of the present invention are shown in the following description of embodiments with reference to the drawings, in which identical reference symbols denote identical or similar components.

### BRIEF DESCRIPTION OF THE DRAWINGS

FIG. 1 shows a tube section as it can be used in a method for producing a bag;

FIG. 2 shows the tube section from FIG. 1 with a formed connection section;

FIG. 3 shows the process of butt welding in a device; and

FIG. 4 shows a schematic view of a bag.

### DETAILED DESCRIPTION

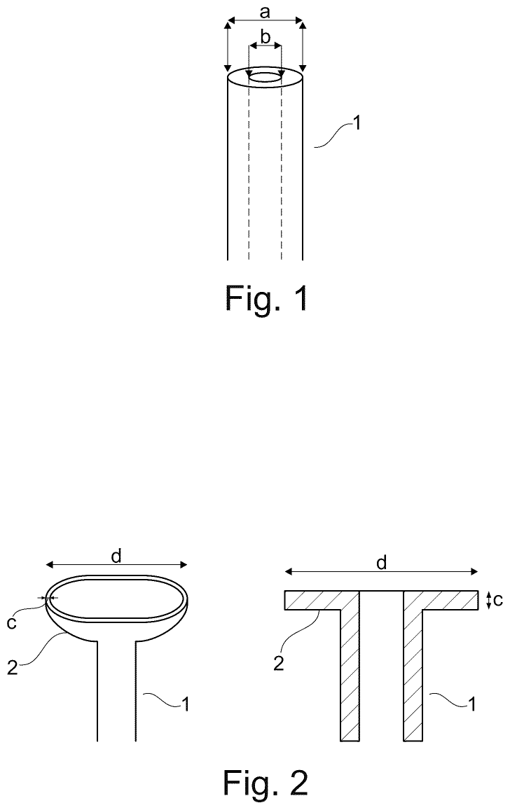

FIG. 1 shows a tube section as it can be used in a method according to the present disclosure.

The tube section can consist of a polymer material and in the initial state can have an outer diameter (length a in FIG. 1) of 5 mm to 10 mm, e.g., of 4.9 mm to 8 mm and an inner diameter (length b in FIG. 1, diameter of the lumen) of 3 mm to 8 mm, e.g., of 3.5 mm to 6 mm.

As shown in FIG. 2, a tulip-shaped or flange-like connection section 2 is formed on the tube section 1 by expanding it under the effect of heat.

A thickness of the connection section 2 in the longitudinal direction of the tube section (length c in FIG. 2) is between 0.1 mm and 2 mm, e.g., between 0.3 mm and 0.5 mm, and e.g., is approximately 0.4 mm.

A diameter of an end face of the flange-like connection section (length d in FIG. 2) is between 5 mm and 25 mm, e.g., between 10 mm and 14 mm, and e.g., is approximately 12 mm.

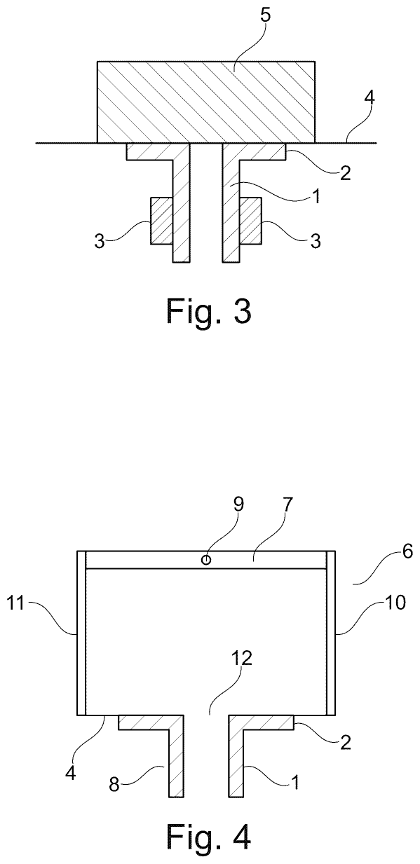

FIG. 3 schematically shows a device during butt welding. The device has a retaining device in the form of two movable retaining jaws 3, between which the tube section 1 is clamped and fixed.

The retaining jaws 3 can completely surround the tube section 1 in their retaining position.

A film 4 is positioned in a receiving section of the device and is in contact with the connection section 2 of the tube section 1.

In this arrangement, the film 4 is positioned between the connection section 2 and a movable and heatable pressure plate 5. The pressure and heat of the pressure plate 5 welds the preheated connection section 2 to the film 4 and bonds it firmly.

FIG. 4 shows a schematic view of a bag 6 according to the present disclosure, which is formed by folding over the film 4 and sealing two outer edges of the film 4 to form a sealing seam 7 on a side opposite the connection 8 formed by the tube section 1 connected to the film 4.

The sealing seam 7 has at least one suspension eye 9. On two opposite sides 10 and 11, the bag 6 is respectively limited by a sealing seam.

In the area of the connection 8, the film 4 has an opening 12 through which the content of the bag 6 can flow outwards through the connection 8.

The opening can be created, for example, by punching out of the film before butt welding the connection or by inserting a hot mandrel into the film 4 after butt welding the connection.

1-14. (canceled)

15. A method for producing a bag for receiving at least one of a medical product or a dry medical concentrate, the method comprising:
providing a flat film with two outer edges;
fixing at least one connection to the flat film using butt welding;
producing a film tube out of the flat film using a forming shoulder and by sealing the outer edges of the flat film together;
producing a sealing seam that extends perpendicular to at least one of a longitudinal direction or extending direction of the film tube, whereby a receiving pocket is produced in the film tube;
filling the receiving pocket along at least one of the longitudinal direction or the extending direction of the film tube with at least one of liquid or solid contents; and
closing the receiving pocket by producing an additional sealing seam that extends perpendicular to at least one of the longitudinal direction or the extending direction of the film tube, whereby the receiving pocket is closed, and a completely closed chamber is formed.

16. The method according to claim 15, wherein filling the receiving pocked comprises filling the receiving pocket in the vertical direction from above.

17. The method according to claim 15, wherein fixing the at least one connection to the flat film by butt welding comprises at least one of the following:
providing a cylindrical tube section;
preforming a connection section of the tube section by locally expanding the tube, whereby a flange-like connection section is formed by means of pressure and heat;
placing the connection section on the flat film so that the flat film is interposed between the connection section and a pressure plate; or
welding the connection section to the flat film by pressing on the connection section while exerting a counter-pressure by means of the pressure plate, whereby the connection section is connected to the flat film and forms a connection.

18. The method according to claim 15, wherein the at least one connection is preformed at a temperature between 80° C. and 300° C.

19. The method according to claim 15, wherein the at least one connection is preformed at a temperature between 220° C. and 280° C.

20. The method according to claim 19, wherein the at least one connection is preformed in a period of time of 0.1 s to 4 s.

21. The method according to claim 19, wherein the at least one connection is preformed in a period of time of 1.5 s to 3.5 s.

22. The method according to claim 19, wherein the at least one connection is preformed at a pressure of 0.1 N/mm² to 2 N/mm².

23. The method according to claim 19, wherein the at least one connection is preformed at a pressure of 0.3 N/mm² to 0.5 N/mm².

24. The method according claim 15, wherein fixing the at least one connection comprises preforming the at least one connection, wherein a flange-like connection section of a tube section is formed during preforming, wherein a thickness of the flange-like connection section in the longitudinal direction of the tube section is between 0.1 mm and 2 mm.

25. The method according to claim 24, wherein the thickness of the flange-like connection is between 0.3 mm and 0.5 mm.

26. The method according to claim 24, wherein the thickness of the flange-like connection is approximately 0.4 mm.

27. The method according to claim 24, wherein a diameter of an end face of the flange-like connection section is between 5 mm and 25 mm.

28. The method according to claim 27, wherein the diameter is between 10 mm and 14 mm.

29. The method according to claim 27, wherein the diameter is 12 mm.

30. The method according to claim 15, wherein fixing the at least one connection comprises placing a connection section on the flat film, wherein a temperature of the connection section is at least partially between 200° C. and 280° C.

31. The method according to claim 15, wherein welding the connection section to the flat film is carried out at a temperature of a pressure plate of 80° C. to 150° C.

32. The method according to claim 31, wherein welding the connection section to the flat film is carried out at a temperature of a pressure plate of 90° C. to 110° C. for a time period of 0.1 s to 4 s.

33. The method according to claim 32, wherein welding the connection section to the flat film is carried out for a time period of 1.5 s to 3.5 s and a pressure of 0.1 N/mm² to 2 N/mm².

34. The method according to claim 33, wherein welding the connection section to the flat film is carried out at a pressure of 0.3 N/mm² to 0.5 N/mm².

35. The method according to claim 15, wherein completely closed chambers are produced continuously along a continuous film tube and are connected to each other by the sealing seam, and the completely closed chambers are separated from each other by separation along a sealing seam extending perpendicular to at least one of the longitudinal direction or extending direction of the film tube, whereby separate bags are produced, each having one or more chambers.

36. The method according to claim 15, wherein
a peel seam extending in the longitudinal direction is produced in the film tube;
the peel seam detachably connects two layers of the film tube to one another; and
two adjacent receiving pockets are produced and separated by the peel seam.

37. The method according to claim 36, wherein each of the two adjacent receiving pockets are filled by two separate filling lances with at least one of a liquid, solid, or pasty content, which is designed for producing dialysate.

38. A device for fixing at least one connection of a film using butt welding, the device comprising:
a retaining device for holding a tube section;
a receiving section;
a movable pressure plate; and
a control unit programmed to control the device to carry out a method comprising:
providing a flat film with two outer edges;
fixing at least one connection to the flat film by butt welding;
producing a film tube out of the flat film by a forming shoulder and by sealing the outer edges of the flat film together;
producing a sealing seam that extends perpendicular to at least one of a longitudinal direction or extending direction of the film tube, whereby a receiving pocket is produced in the film tube;
filling the receiving pocket along the longitudinal direction of the film tube with at least one of liquid or solid contents; and
closing the receiving pocket by producing an additional sealing seam that extends perpendicular to the longitudinal direction of the film tube, whereby the receiving pocket is closed, and a completely closed chamber is formed.

39. The device of claim 38, wherein the retaining device comprises a flange-like connection section.

40. The device of claim 38, wherein the receiving section is configured to receive the film and position the film in contact with a connection section.

41. The device of claim 38, wherein the pressure plate is heatable.

42. The device according to claim 38, wherein the control unit is programmed to move and control at least one of the retaining device or the pressure plate relative to each other in such a way that a connection section held in the retaining device is pressed together with a film positioned in the receiving section for a certain period of time and at a certain temperature and pressure, so that the connection section is butt-welded to the film and forms a connection.

43. A bag which has at least one side formed by a folded-over film to which at least one connection is non-detachably arranged using butt welding, wherein the bag is produced by a method comprising:
providing a flat film with two outer edges;
fixing at least one connection to the flat film by butt welding;
producing a film tube out of the flat film by a forming shoulder and by sealing the outer edges of the flat film together;
producing a sealing seam that extends perpendicular to at least one of a longitudinal direction or extending direction of the film tube, whereby a receiving pocket is produced in the film tube;
filling the receiving pocket along the longitudinal direction of the film tube with at least one of liquid or solid contents; and
closing the receiving pocket by producing an additional sealing seam that extends perpendicular to the longitudinal direction of the film tube, whereby the receiving pocket is closed, and a completely closed chamber is formed.

44. The bag according to claim 43, wherein the at least one connection comprises a tube section.

45. The bag according to claim 43, comprising at least two chambers, which are separated from each other by an openable peel seam.

46. The bag according to claim 43, wherein the bag does not have, in addition to the at least one connection arranged on the bag by butt welding, a filling port designed to fill the bag or at least one chamber thereof with a desired content at the factory.

47. A bag system for peritoneal dialysis with a solution bag and a drainage bag, wherein the solution bag and the drainage bag are connected to a common three-way valve, wherein at least one of the solution bag or the drainage bag is a bag according to claim 43.
